(19) United States
(12) Patent Application Publication
(10) Pub. No.: US 2013/0006293 A1
(43) Pub. Date: Jan. 3, 2013

(54) INTERNALLY IMPLANTABLE NASAL STENT

(76) Inventor: Oakley SMITH, Toronto (CA)

(21) Appl. No.: 13/432,396

(22) Filed: Mar. 28, 2012

Related U.S. Application Data
(60) Provisional application No. 61/502,952, filed on Jun. 30, 2011.

(51) Int. Cl. A61M 29/00

(52) U.S. Cl. 606/199

(57) ABSTRACT

A nasal stent is provided as a lattice formed from a biologically absorbable material. The lattice has a plurality of intersecting semi-rigid rods defining interstitial spaces therebetween. The interstitial spaces are sufficiently large such that, when the stent is installed between a patient's overlying mucosal lining and underlying cartilaginous septum, the stent does not substantially impede diffusion of blood supply from the overlying mucosal lining to the underlying cartilaginous septum. The lattice also has sufficient rigidity so as to be able to compress the septal cartilage into a flat plane.

### CROSS-REFERENCE TO RELATED APPLICATIONS

This application claims priority to U.S. Provisional No. 61/502,952 filed Jun. 30, 2011, the contents of which are incorporated herein by reference.

### FIELD OF INVENTION

The invention relates to the field of surgical aids, and more particularly to stents in aid of septoplasty procedures.

### BACKGROUND OF INVENTION

Septoplasty is a surgical procedure for correcting an abnormality or deformity of the nasal septum. The septum is the central common wall of the nose separating the two nostrils and creating two nasal channels. The deformity is typically characterized by a crookedness, curvature or deviation of the septum that substantially blocks the opening to the nasal passages preventing normal nasal breathing or causing symptoms of difficulty breathing clearly through the nose. FIG. 1 schematically illustrates a normal septum and FIG. 2 schematically illustrates a deformed septum requiring a septoplasty, which is a commonly performed procedure to straighten this wall.

The surgical goal of septoplasty is to create a straight septum and restore its position to the nasal midline. About 40% of these surgeries are successful, 40% partially successful, and 20% could be considered failures, with the more severe deformities characterized by higher failure rates. There is also a significant (greater than 5%) surgical revision rate.

Septoplasty typically involves separating the mucosal covering from each side of the cartilaginous septum and performing surgical manipulations on the cartilage. There are a variety of techniques to create a straighter septum. These techniques include freeing the septum from its surrounding soft tissue connections, partial removal of septal cartilage, scoring the cartilage, shifting the cartilage, adding cartilage, suturing the cartilage and partially crushing the cartilage. Depending on the abnormality of the septum, these manipulations have varying degrees of success.

After the septal manipulations are completed, the mucosal covering are laid back against the septum and held in place for sufficient time for healing. Techniques to achieve this include suturing the coverings to the septum, stuffing the nasal channels with packing, placing a plastic sheet(s) in the nasal channels and transfixing the sheet(s) to the septum with sutures.

Stabilization plates are sometimes used in septoplasty procedures. In cases of revision surgery or severely damaged or traumatized septums, the septum can be found in several fragments that are surgically removed from the body. These fragments of cartilage then have to be reconnected to form a straight plate. At other times there is a lack of cartilage and new cartilage is harvested from elsewhere in the body and added and incorporated with the remaining septal cartilage. This can be achieved by suturing them together or using a flexible biocompatible plate, such as the PDS™ Flexible Plate marketed by Ethicon Inc. as a framework to stabilize and support the septal fragments during healing. The newly recreated straight septum, with or without a biocompatible plate, is returned to its initial location between the mucosal coverings and the plate, if present, Is eventually absorbed by the body, eliminating the need for a removal procedure.

However, these stabilization plates have limitations on their use and are used for the mostly severely damaged cases representing only a small percentage of septoplasties.

### SUMMARY OF INVENTION

The chief limitation of the prior art stabilization plates is that they cannot be used on both sides of the septum, limiting the range of surgical techniques. If used on both sides of the septum, the prior art stabilization plates could cut off the blood supply to the septum with death and eventual reabsorption and loss of the septum. This prevents their use in a vise like manner to straighten a septum by compressing the bend, deformed or twisted sections of the septum. Furthermore the flexibility of the plate limits its ability to fully straighten bent, deformed or twisted septal areas.

According to one aspect of the invention a nasal stent is provided as a lattice formed from a biologically absorbable material. The lattice has a plurality of intersecting semi-rigid rods defining interstitial spaces therebetween. The interstitial spaces are sufficiently large such that, when the stent is installed between a patient's overlying mucosal lining and underlying cartilaginous septum, the stent does not substantially impede diffusion of blood supply from the overlying mucosal lining to the underlying cartilaginous septum. The lattice also has sufficient rigidity so as to be able to compress the septal cartilage into a flat plane.

The lattice preferably also includes a plurality of lands integral with or connected to the lattice. These lands may be used as suturing points. The lands are of sufficiently small cross-sectional area so as to not substantially impede the blood supply from the patient's overlying mucosal lining.

According to another aspect of the invention, a septoplasty method is provided. The method Includes:

(i) provisioning first and second nasal stents, each stent comprising a lattice formed from a biologically absorbable material, the lattice having a plurality of intersecting semi-rigid rods defining interstitial spaces there between, the interstitial spaces being sufficiently large so that, when the stent is installed between a patient's overlying mucosal lining and underlying cartilaginous septum, the stent does not substantially impede diffusion of blood supply from the overlying mucosal lining to the underlying cartilaginous septum;

(ii) accessing the patient's cartilaginous septum and scoring, shifting, crushing, cutting or otherwise manipulating the cartilaginous septum to enable it to be straightened and brought to the nasal midline;

(iii) installing the first stent in the patient's first nostril between the mucosal lining thereof and the manipulated cartilaginous septum;

(iv) installing the second stent in the patient's second nostril between the mucosal lining thereof and the manipulated cartilaginous septum; and

(v) compressing together the mucosal lining of the first nostril, the first stent, the cartilaginous septum, the second stent and the mucosal lining of the second nostril by sutures.

The surgical application of two compressive stents does not require the removal of the septum or parts thereof from the body. This reduces the degree of invasiveness of surgery and avoids the potential complications arising from removing the septum or parts thereof from the body.

### BRIEF DESCRIPTION OF DRAWINGS

The foregoing and other aspects of the invention will be better understood with reference to the drawings, wherein:

FIGS. 1 and 2 are schematic illustrations of an abnormal nonlinear septum and normal (straight) septum, respectively;

FIG. 3 is a front view of a nasal stent according to a preferred embodiment of the invention;

FIG. 4 is a rear view of the preferred nasal stent;

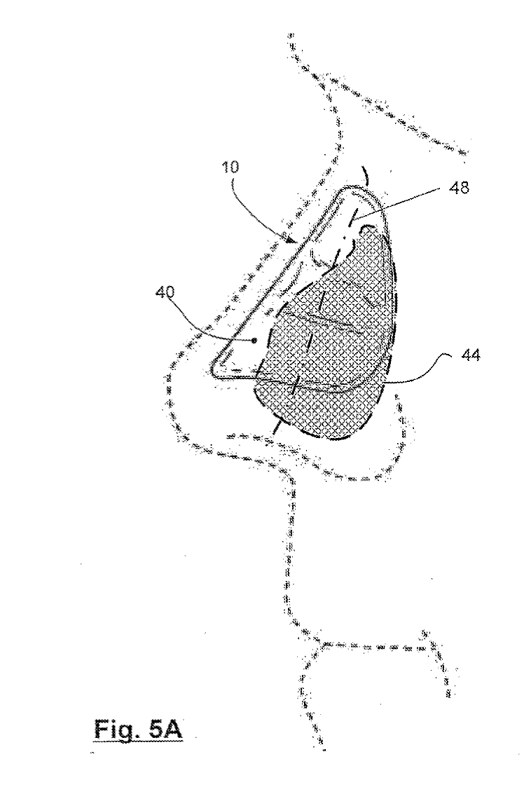

FIGS. 5A, and 5B are perspective view diagrams illustrating the use of the preferred nasal stent according to a preferred surgical technique; and

FIG. 6 is a cross-sectional view diagram illustrating the use of the preferred nasal stent according to the preferred surgical technique.

### DETAILED DESCRIPTION OF PREFERRED EMBODIMENTS

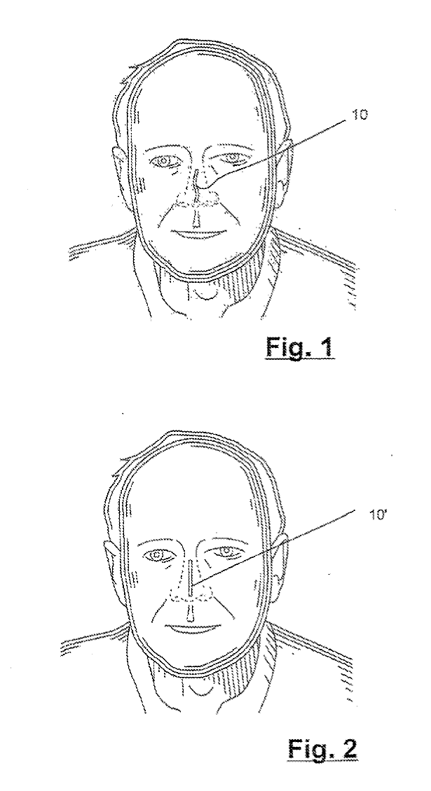

FIG. 2 shows a patient with a deviated septum 10. FIG. 1 shows the desired end result of the septoplasty—a healed straight septum 10′. It Is generally preferable to carry out the septoplasty corporeally In such a manner that the patient bears no visible facial scars, or where scarring is limited to the base of the nose.

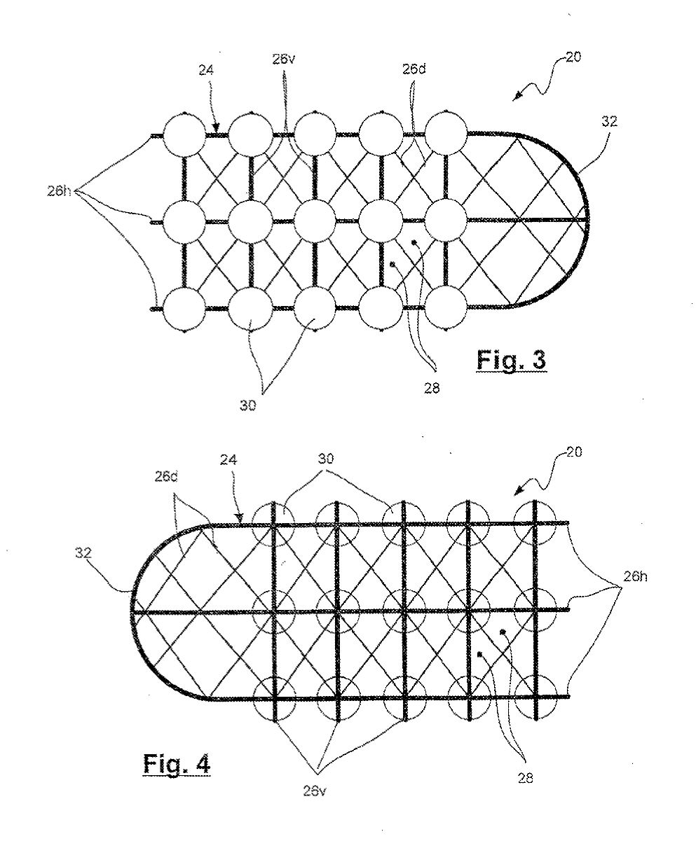

FIG. 3 shows an absorbable and implantable nasal stent 20. Its purpose is to create a straight septum, and more particularly straight septal cartilage. The septal cartilage is typically situated at the narrowest part of the nasal cavity and is responsible for the majority of resistance to airflow, so any curvature, crookedness or other nonlinearity of the septal cartilage can be problematic and effect a patient's breathing. Of course, the stent 20 can be applied to other parts of the septum such as the bony septum.

The stent 20 accomplishes its task by causing the bent or nonlinear areas of the septum to become straight in two different ways. First, the stent 20 acts as a semi-rigid plane to which the bent septum is tied to, enabling the surgeon to compress the septum into a straight position. Second, the stent 20 temporarily supports the surgically weakened septum by acting as a scaffold.

The stent 20 is formed from a biocompatible absorbable material such as poly-p-dioxanone and features a framework or lattice 24 of semi-rigid thin wires or rods 26 with lands 30 located at the intersection points of the semi-rigid rods. More particularly, the illustrated embodiment incorporates three rods 26h running in a horizontal direction and five rods 26v running in a vertical direction. The horizontal and vertical rods 26 are preferably circular in cross-section having a diameter of about 1 mm, but other cross-sectional profiles and thicknesses are possible.

The illustrated embodiment also features a plurality of diagonally running rods 26d that add additional stiffness to the stent 20. These rods 26d are a little thinner in cross-section, e.g., 0.5 mm.

In practice, there is no special affinity to the arrangement of the rods 26 which perform the function of a structural framework. For example, the lattice 24 may be formed only by right and left diagonally oriented rods (i.e., forming a diamond hatch pattern) or only by perpendicularly arranged rods (i.e., forming a square hatch pattern). However, it is important to understand that the lattice should provide considerable interstitial space 28—much more than mere perforations—as will be discussed in greater detail below. For example, individual interstitial spaces can have an area at least 25 mm², and more preferably at least 90 mm².

The lands 30 are situated at the intersections of the rods 26. The lands 30 are preferably provided in the form of small circular plates overlying the rods. The lands 30 provide sufficient surface area for suturing cartilage or tissue to the stent without significantly impacting the interstitial spaces 28. The lands 30 have a thickness in the range from a few hundreds of an inch to about 0.5 mm, more preferably in the range of about 0.05 mm to 0.25 mm, and most preferably in the range of about 0.1 mm to 0.2 mm. The lands 30 are limited in their areas, having a maximal area of about 70 mm², and more preferably at most about 24 mm².

In practice, the stent 20 may be manufactured by molding processes as known in the art per se, wherein the lattice 24 and lands 30 are formed simultaneously in the mold. The rigidity of the forming material such as poly-p-dioxanone may be set during the manufacturing process as required to achieve the function of providing a suturable, structural framework for septoplasty as discussed in greater detail below. Alternatively, the lattice 24 and lands 30 may be formed separately and the lands 30 thereafter affixed to the lattice using a biologically compatible adhesive such as 2-octyl-cyanoacrylate, or mechanically attached to the lattice with suture or the like.

In alternative embodiments the lands 30 may be omitted in their entirety and the stent 20 affixed to tissue by looping suture around the lattice rods.

Overall, the illustrated lattice 24 has a length and a width of about 50 mm by 30 mm. Other sizes are also possible, for example 45 mm by 20 mm. The outer horizontal rods preferably form a semi-circular posterior edge 32 of the stent 20 for making it easier to insert the stent 20 in the nostril.

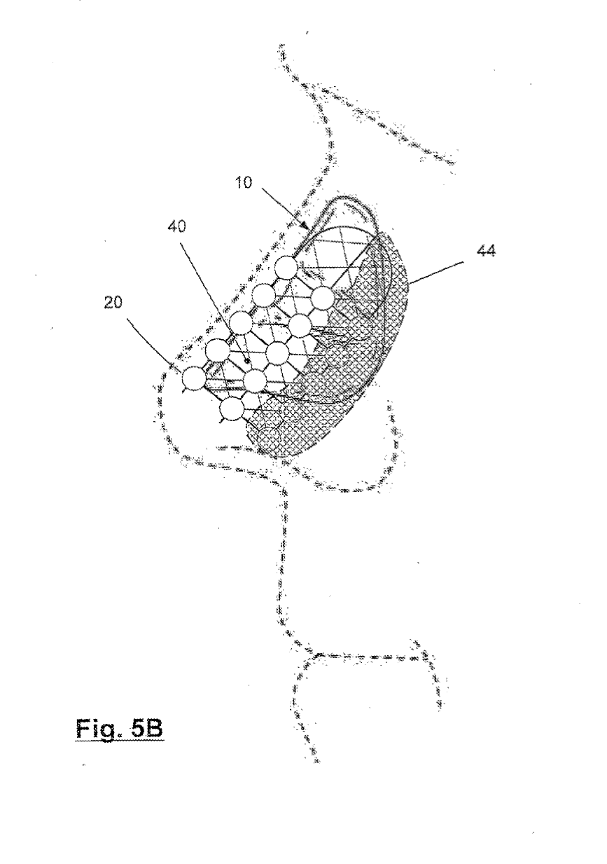

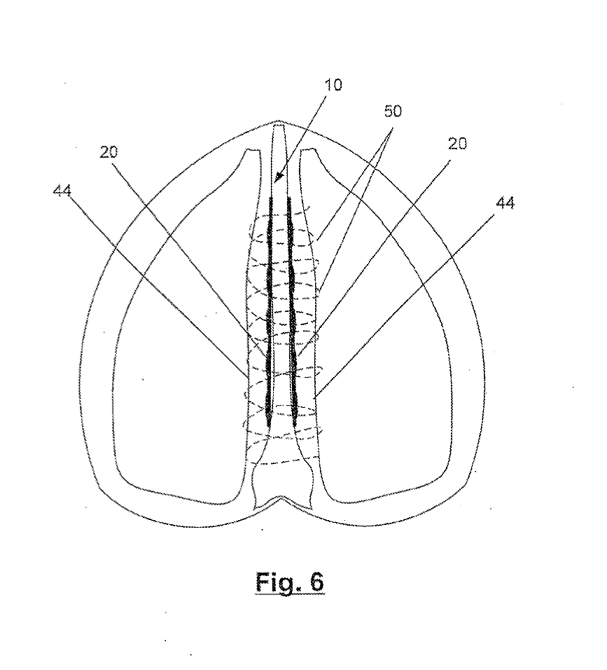

Referring additionally to FIGS. 5A, 5B and 6, the surgeon can use the stent 20 to straighten non-linear septal cartilage 40. First, the mucosal lining or covering 44 in both nostrils is exposed (e.g., via incision 48) to provide access to the septal cartilage 40. This cartilage 40 is scored, shifted, crushed, cut or otherwise manipulated so that it can be straightened. Then, as seen best in the cross-sectional view of 6 a stent 20 is placed in each nostril on each side of the septum 10, between the septum 10 and its mucosal covering 44. Each stent 20 is trimmed as necessary to match the operative area. This sandwich of mucosal covering 44, first stent 20, septum 10, second stent 20 and mucosal covering 44 is held compressed together by sutures 50 placed through the lands 30 or alternatively by suturing around the rods 26 of the lattice 24. The rigid quality of the lattice 24 fixes the septum 10 in the midline and when the first and second stents 20 are sutured together they compresses the septum 10 into a flat structure. The stents 20 will slowly and eventually be absorbed by the body, but the stents will retain sufficient rigidity before they degrade to allow for the creation of collagenous scar tissue by the healing process which will stabilize the septum in this new, midline, flat position. The stent 20 also stabilizes any free pieces of septum by holding them in fixed relationship with each other.

Unlike the prior art the stent 20 can be placed between the mucosal lining 44 and the cartilaginous septum 40 in both nostrils simultaneously. The cartilaginous septum 44 receives its blood supply and excretes waste via diffusion from the overlying mucosa 44, and thus having sufficiently large interstitial spaces 28 and lands 30 of sufficiently small area enables the cartilaginous septum 44 to survive. This characteristic, coupled with the relative rigid nature of the stent 20, enables the foregoing surgical technique to create a straighter septum than would be possible without its use.

The use of the stent 20 is not limited to the above surgical technique. The stent 20 can be used more generally to fix and stabilize any other nasal tissue or bone. Those skilled in the art will understand that a variety of modifications may be made to the preferred embodiments discussed herein without departing from the spirit of the invention.

1. A nasal stent, comprising a lattice formed from a biologically absorbable material, the lattice having a plurality of intersecting semi-rigid rods defining interstitial spaces therebetween, the interstitial spaces being sufficiently large such that, when the stent is installed between a patient's overlying mucosal lining and underlying cartilaginous septum, the stent does not substantially impede diffusion of blood supply from the overlying mucosal lining to the underlying cartilaginous septum, the lattice having sufficient rigidity so as to be able to compress septal cartilage into a flat plane.

2. A nasal stent according to claim 1, wherein the interstitial spaces each have an area of at least 90 mm².

3. A nasal stent according to claim 2, wherein the Interstitial spaces each have an area of at least 25 mm².

4. A nasal stent according to claim 1, including a plurality of lands integral with or connected to the lattice, the lands being of sufficiently small cross-sectional area so as to not substantially impede the blood supply from the patient's overlying mucosal lining.

5. A nasal stent according to claim 4, wherein the lands each have an area of at most 70 mm².

6. A nasal stent according to claim 5, wherein the lands each have an area of at most 24 mm².

7. A nasal stent according to claim 4, wherein the lands are disposed at intersections of the rods.

8. A septoplasty method, comprising:
provisioning first and second nasal stents, each stent comprising a lattice formed from a biologically absorbable material, the lattice having a plurality of intersecting semi-rigid rods defining interstitial spaces there between, the interstitial spaces being sufficiently large so that, when the stent is installed between a patient's overlying mucosal lining and underlying cartilaginous septum, the stent does not substantially impede diffusion of blood supply from the overlying mucosal lining to the underlying cartilaginous septum;
accessing the patient's cartilaginous septum and scoring, shifting, crushing, cutting or otherwise manipulating the cartilaginous septum to enable it to be straightened and brought to the nasal midline;
installing the first stent in the patient's first nostril between the mucosal lining thereof and the manipulated cartilaginous septum;
installing the second stent in the patient's second nostril between the mucosal lining thereof and the manipulated cartilaginous septum; and
compressing together the mucosal lining of the first nostril, the first stent, the cartilaginous septum, the second stent and the mucosal lining of the second nostril by sutures.

9. A method according to claim 8, wherein the interstitial spaces each have an area of at least 25 mm².

10. A method according to claim 9, wherein the interstitial spaces each have an area of at least 90 mm².

11. A method according to claim 8, including provisioning plurality of lands integral with or connected to the lattice, the lands being of sufficiently small cross-sectional area so as to not substantially impede the blood supply from the patient's overlying mucosal lining, and suturing through the lands.

12. A method according to claim 11, wherein the lands each have an area of at most 24 mm².

13. A method according to claim 11, wherein the lands each have an area of at most 70 mm².

14. A method according to claim 11, wherein the lands are disposed at intersections of the rods.
